(19) United States
(12) Patent Application Publication
(10) Pub. No.: US 2005/0137585 A1
(43) Pub. Date: Jun. 23, 2005

(54) Back loading endoscopic instruments

(76) Inventors: Jamie Landman, St. Louis, MO (US); Walter N. Ryan, Bloomington, IN (US); Troy W. Wingler, Gosport, IN (US); Thomas L. Foster, Poland, IN (US)

Correspondence Address: BRINKS HOFER GILSON & LIONE/CHICAGO/COOK, PO BOX 10395, CHICAGO, IL, 60610, US

(21) Appl. No.: 11/020,328

(22) Filed: Dec. 22, 2004

Related U.S. Application Data
(60) Provisional application No. 60/532,397, filed on Dec. 23, 2003.

(51) Int. Cl. A61B 17/00

(52) U.S. Cl. 606/1; 600/562; 606/27; 606/45

(57) ABSTRACT

A back loading instrument for use with a medical device, such as an endoscope or a colonoscope, features larger endoscopic instruments or end effectors and enables gathering of much larger samples, such as biopsies. The instrument also allows for superior grasping, mobilization, cutting, or removal of tissue or foreign bodies from a patient. The end effector, such as a biopsy forceps, of the back loading instrument has a cross-sectional area larger than that of the working channel of the endoscope or other medical device used to control the instrument. By using this medical device as part of a minimally-invasive procedure, trauma to the patient is minimized.

This application claims the benefit of the filing date under 35 U.S.C. § 119(e) of Provisional U.S. Patent Application Ser. No. 60/532,397, filed on Dec. 23, 2003, which is hereby incorporated by reference in its entirety.

### FIELD OF THE INVENTION

The field of the invention is that of medical and surgical instruments, and in particular, medical and surgical instruments intended primarily for minimally-invasive procedures. These instruments typically have a small diameter for entering body spaces or orifices of limited size.

### BACKGROUND

One trend in modern surgery is the trend toward minimally-invasive procedures, in which laparoscopic or endoscopic procedures are used. These procedures tend to be less invasive to the patient, using a body orifice or a puncture into a body cavity or a natural existing space as an entry point for a medical device. Examples of entry points include a component of the urinary tract or the gastrointestinal system. These procedures are used in order to avoid major incisions for surgical access, accomplishing the medical procedure in less time with less risk to the patient, and with reduced patient convalescence.

Minimally invasive procedures thus provide benefits to the patient, in the sense that the procedures may be accomplished more quickly and more economically, and with less discomfort and less overall invasion of the body when compared to traditional open surgical techniques. Minimally-invasive procedures are not without their problems, however. One significant problem is that body orifices tend to be small, and thus minimally invasive surgical procedures may be difficult to accomplish due to the very small access provided. For example, when entering the urinary tract, space in the urethra and the ureter is very limited, since these passages are only several millimeters in diameter. As a result, the majority of minimally invasive procedures are performed with an endoscope, or with instruments bearing some resemblance to an endoscope, such as a hysteroscope or a ureteroscope. These instruments typically have a working channel that limits instruments or surgical tools to a diameter of about 3 Fr (1 mm).

The size of the surgical tools is limited because they are routed to the operating site through the working channel of the endoscope or similar instrument. The working channel may vary from about 2.6 Fr to about 3.4 Fr, about 1 mm in diameter. To date, the size of the surgical tools that can be used with endoscopic equipment has been limited because the instrumentation is routed to the operating site through the working channel of the endoscope or similar instrument. These very small instruments can be challenging to design and build. Using these instruments is also difficult because the small size of the end-effector is not always effective to accomplish surgical goals, which may include grasping tissues, taking biopsy samples, or moving tissues. Engineering and materials limitations become very evident when instruments of such small diameters are created.

For the majority of minimally invasive procedures, the user accesses the desired site, perhaps with a wire guide, and then uses one or more wire guides, and perhaps a sheath, for greater access. Because the working instruments are deployed via the working channel of the endoscope being used to access the target site, it is frequently necessary to make repeated trips to the target location to achieve the desired end-point of the operation, such as the grasping of structures, biopsy of tissues, or manipulation of either tissues or structures. A tremendous improvement in current technology for minimally invasive surgery would be instruments or end-effectors which are not limited to the diameter of a working channel of existing endoscopes. What is needed is a surgical instrument which allows more freedom to operate while adhering to the principles of minimally-invasive surgery.

### BRIEF SUMMARY

There are many aspects and embodiments of the invention. One aspect of the invention is a back-loading medical instrument comprising a flexible hollow tube, a control rod within the hollow tube, and a distal functional portion. The functional portion is attached to at least one of the flexible hollow tube and the control rod, and has a cross-sectional area larger than the tube and larger than a working channel of a therapeutic or diagnostic medical device for use with the back-loading medical instrument. This embodiment allows for large laparoscopic end-effectors deployed through a single, slightly larger incision, to be attached in the body cavity to smaller diameter control rods. Such smaller diameter rods may be passed through diminutive access sites for what may be termed very-minimally invasive access.

Another aspect of the invention is a back loading instrument for an endoscope. The instrument comprises a distal portion including a forceps, a control portion operatively connected to the forceps, and a proximal portion for insertion into a working channel of an endoscope. The cross-sectional area of the forceps is greater than a cross-sectional area of a working channel of the endoscope.

Another aspect of the invention is a back loading endoscopic biopsy instrument. The instrument comprises a flexible hollow member having a proximal end and a distal end, and a flexible inner member having a proximal end and a distal end and extending through said hollow outer member. The instrument also comprises a jaw assembly having a pair of jaws extending distally, the jaw assembly coupled to at least one of the hollow outer member and the flexible control member. In this embodiment, a circumference of the outer jaws in a closed state is greater than a circumference of a working channel of the endoscope.

Another aspect of the invention is a method of making a back loading endoscopic instrument. The method comprises forming an openable functional portion, a flexible inner member, and a hollow tubular member, and assembling the hollow tubular member outside the flexible inner member. The method also comprises attaching the functional portion to at least one of the flexible inner member and the hollow tubular member. In this embodiment, an outer periphery of the openable functional portion is greater than an outer periphery of a working channel of an endoscope when the functional portion is in a closed state.

There are many ways to practice the present invention, a few of which are shown in the following drawings and specification. The embodiments are not meant to limit the invention, but rather to describe and illustrate a few of the many ways that the present invention may be used.

### BRIEF DESCRIPTION OF THE DRAWINGS

FIG. 1 is a perspective view of an embodiment of a back-loading biopsy grasper mounted on a coil;

FIG. 2 is a closer view of a back loading endoscopic forceps showing the relationship with a working channel of an endoscope;

FIG. 3 is an embodiment of a back loading instrument with biopsy cups;

FIG. 4 is another embodiment of a back loading instrument, an angled forceps;

FIG. 5 depicts a back loading instrument with hot biopsy forceps;

FIG. 6 depicts a back-loading instrument with a sharp cutting tool and a spike;

FIG. 7 depicts a back loading forceps preferably deployed by retracting a sheath rather than actuating a distal control mechanism; and

FIGS. 8-10 depict additional embodiments of the back-loading instrument.

FIGS. 11-14 depict handles suitable for use in controlling the back-loading endoscopic devices.

### DETAILED DESCRIPTION OF THE DRAWINGS AND THE PRESENTLY PREFERRED EMBODIMENTS

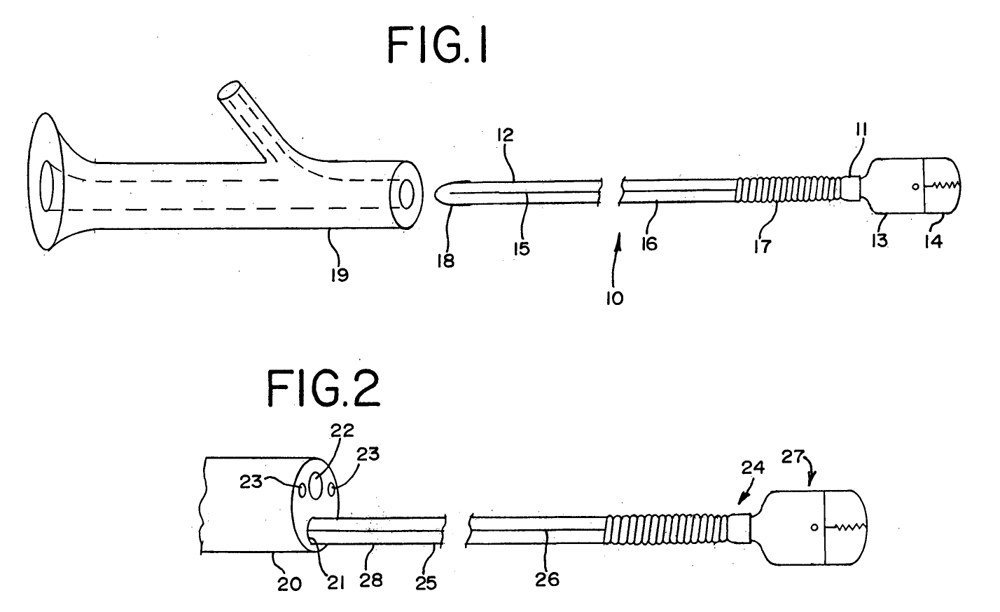

Several embodiments of the back-loading instruments are presented and explained in this portion. FIG. 1 depicts a back-loading biopsy grasper 10 for back loading into an endoscope 19. Grasper 10 includes a distal portion 11 and a proximal portion 12, with a grasper control portion 13 and upper and lower jaws 14. Control portion 13 may include pins, levers and connections for a control cable 15 and a flexible outer sheath 16 for the back loading biopsy grasper. Jaws 14 preferably do not extend more than about 1 cm from an endoscope or other diagnostic or therapeutic instrument with which the back-loading grasper is used.

The distal portion 11 may be as large as needed, but the distal portion will still have to be inserted into the patient, for example, through an access sheath into which the distal portion will fit. It is also preferred that the diameter of distal portion 11 not be so large as to interfere with light from the scope that is transmitted to the site of interest, or from light from the site reflected back into the scope for the physician or other health professional to see.

The outer sheath 16 is desirably not more than about 2.6 Fr, and may be as large as 3.4 Fr, in diameter, so that the proximal portion 12 of the back-loading grasper may be inserted into the distal end of a diagnostic or therapeutic instrument. The medical device may include an endoscope, a colonoscope, a bronchoscope, or a ureteroscope. In some embodiments, the outer sheath interacts with the forceps or other functional portion on the distal end of the endoscopic instrument. These functional portions may include any forceps, grasper, incisor, extractor, cutting instrument, or end-effector that may be desired. The larger end-effectors may have additional capabilities, such as a biopsy forceps for taking tissue specimens that also has an active electrosurgical component for coagulation ( a “hot” biopsy forceps).

The functional portions, or end-effectors, may be made by any convenient manufacturing operations. They may be made by stamping or blanking from sheet metal and forming into the desired shapes, as in a process using progressive dies. For smaller volumes, they may be machined as desired and the movable jaws formed on an axis supplied by a rivet or other fastener. Alternately, the jaws may be formed from tubing by an electrical discharge machining (EDM) process, with or without additional forming. Forceps, extractors, incisors, or other desired functional distal portions may also be formed by machining stainless steel plate or other desired material. Components for end effectors may also be cast, and like other processes, the parts assembled, if necessary, into an articulable end effector.

The control cable 15 may be made from any convenient, flexible and strong material, such as stainless steel or a shape-memory alloy, such as Nitinol®. The cable should not elongate in use and may be made of a single wire or may be a bundle of smaller wires. The sheath 16 is ideally also strong and very flexible. It may be made from a polymer, such as a fluoropolymer or a polyimide-type polymer. Other polymers may also be used, such as PEEK (polyetheretherketone). The cable can be of very small diameter, allowing for minimal obstruction of the working channel while accomplishing deployment of the end-effector, i.e., opening and closing the jaws of an instrument.

Distal portion 11 may be attached to a flexible coiled wire 17 surrounded by the sheath, the coiled wire extending through to the proximal end for connection to endoscope 19. The outer diameter of the sheath, as mentioned above, preferably is about 2.6 to about 3.4 Fr, or about 1 mm, in diameter. The control cable 15 inside the coil should not bind or chafe on the coil. When back-loading the control cable, coil, and sheath into the endoscope or other diagnostic of therapeutic instrument, the proximal ends should be shielded, perhaps by a soft cap or soft plastic end 18, so that the ends do not scratch or damage the endoscope.

FIG. 2 depicts a distal end 20 of an endoscope which may be used with any of the back-loading instruments of the present invention. The endoscope may include a working channel 21, a lens 22, and one or more fiberoptic light sources 23. The proximal or back end 28 of the back-loading endoscopic instrument 24 is inserted into the working channel 21 on the distal end 20 of the endoscope. As discussed above, there are many types of endoscopic instruments which may be designed for back loading. The embodiment in FIG. 2 includes a grasper 27 controlled by an outer sheath 25 and a control rod 26.

In use, the surgeon may maneuver the endoscope to the area of interest within a patient. The endoscope includes a forceps or other tool on the distal end, along with a fiberoptic light source and lens, and a camera, or optical transmitter (not shown), to generate and transmit images through the endoscope. A forceps is an endoscopic tool used for grasping a structure, such as tissue, for maneuvering or for removal. Using the images from the endoscope, the surgeon maneuvers the endoscope and then the forceps to the desired position. Typically, a biopsy sample is then acquired and removed from the patient. Using the back-loading endoscopic tool, a much larger sample, more than the usual microscopic size, may be taken. This enables the pathologist or laboratory technician to more easily analyze the sample. A prognosis or a treatment may then be decided more easily.

FIG. 3 depicts another embodiment of a back-loading endoscopic cup. The back loading endoscopic cup 30 includes a hollow urethane tube and a control wire 37 for controlling the cup. Urethane is tough and strong, as well as flexible, and may be formulated to be smooth and with a low coefficient of friction against control wire 37. The distal portion 35, which may be metallic, is mounted to tube 38 and includes first and second jaws 31, 32, control linkages 33, 34 for the jaws, and a control portion 35. The control portion and jaw portions have a larger diameter than that of tube 38. Tube 38 is preferably about 1 mm outer diameter, preferably about 2.4 to 2.6 Fr. The control rod may preferably be stainless steel or Nitinol®. Tube 38 may be coated with a thin, smooth, lubricious coating 39, such as Teflon® or other fluoropolymer, or other suitable smooth coating. A very thin film, such as from a fluoropolymer or PEEK, may be used instead.

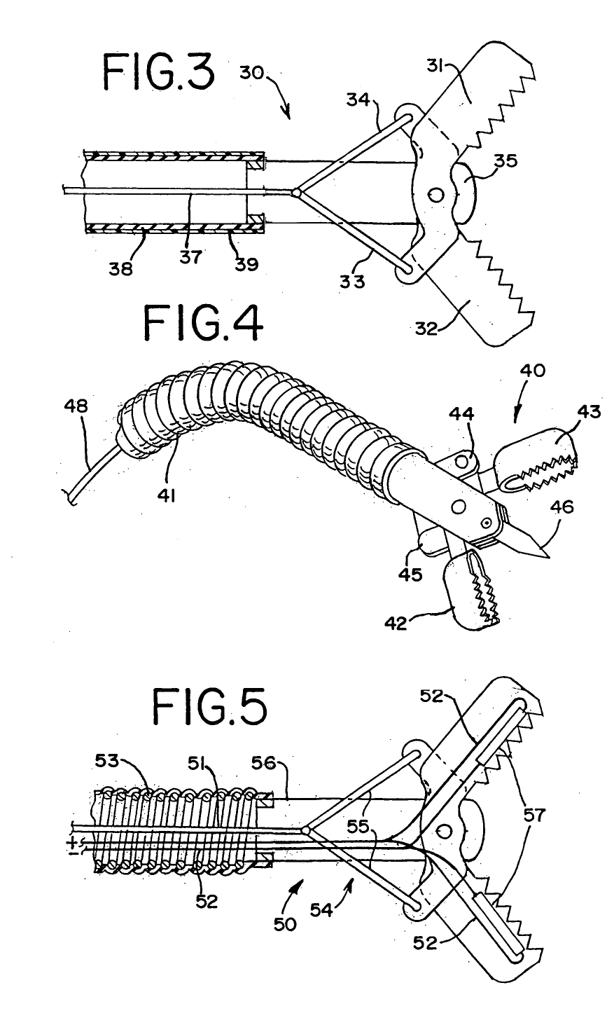

The back loading instruments seen so far have largely been straight-forward, i.e., the deployed instruments, the cup or graspers or forceps, are in line with the endoscope and the sheath. FIG. 4 depicts an embodiment in which the back-loading instrument, and the coil and control cable, may be at an angle to the advance of the endoscopic instrument. FIG. 4 depicts an angle forceps 40, with first and second jaws 42, 43, control portions 44, 45, and a spike 46. The angle forceps 40 is angled at about 45° to the coil 41 and control cable 48. The sheath and control cable may be made from shape memory alloys, such as Nitinol®.

Nitinol® forming techniques are used so that the control cable and coil will have a tendency to assume the shape in which they were trained. As is well known to those skilled in shape memory alloys, the sheath and the control cable may be formed in the desired shape, perhaps with forms or tools. They are formed at or below room temperature, and are then heat treated at a high temperature, typically from about 400° C. to about 500° C. Thus, the natural tendency of the angled forceps is to assume a position at about a 45° angle to the sheath and the control rod. This particular forceps allows the surgeon one more degree of freedom in diagnosing a patient or in conducting a biopsy, i.e. obtaining a desired sample for pathology.

Another embodiment is a hot biopsy forceps. The hot biopsy forceps is similar in concept to other back-loading instruments, but the forceps include heating elements integral to the forceps. Using the heating elements, the jaws of the forceps can be quickly heated to help free a biopsy sample or to coagulate small bleeding sites related or not related to the biopsy. When the sample has been obtained, the electric power may be turned off, and the jaws will quickly cool off to avoid any trauma to other nearby tissues.

The hot biopsy forceps 50 is depicted in FIG. 5. The hot biopsy forceps includes a control rod 51, one or more wires 52 to conduct electricity, and a coil 53. The forceps has a control portion 54 at the distal end, connected to coil 53 and including linkages 55 for control rod 54 to jaws 56. The jaws 56 may include resistance heating elements 57 connected by wires 52. The heating elements have much lower electrical resistance than the steel typically used for forceps jaws.

The surgeon maneuvers the endoscope into the desired area, such as the gastrointestinal tract. Biopsies in this area may include polyps. The hot biopsy forceps may include one or more heating elements, such as one heating element in one jaw and an optional heating element in the other jaw. The current may be bipolar, as in alternating or direct current electricity, or monopolar current may be used. The surgeon maneuvers the jaws in place around the desired sample and closes the jaws. Current is applied for a period sufficiently long to free the sample from its environment and to retrieve the sample inside the jaws for subsequent analysis.

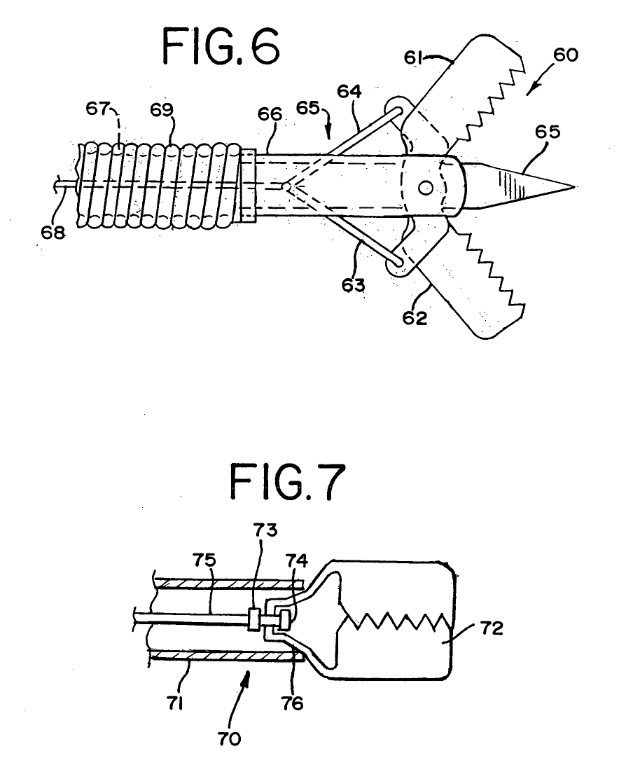

FIG. 6 depicts another embodiment of a back-loading instrument, a forceps with a sharp cutting tool and a spike. In this embodiment, the back loading forceps 60 constitutes the distal portion of a the instrument and includes an upper jaw 61, a lower jaw 62, a control linkage 63 for controlling upper jaw 61, a control linkage 64 for controlling lower jaw 62, a spike 65, and a control portion 66 for connecting control linkages 63, 64 to control cable 68. Control cable 68 leads back through sheath 67. In this embodiment, sheath 67 may be a coiled wire spring with a smooth coating 69, such as a Teflon® coating. The outer diameter of sheath 67 preferably is about 2.4 to 2.6 Fr or narrower, and the inner diameter is sufficient so that control cable 68 does not bind or chafe during operation of the forceps. In the embodiment depicted in FIG. 6, the surgeon may maneuver the forceps to impale the spike 65 on a tissue or sample of interest. The surgeon then operates the forceps to sever the sample and remove it from the patient for later pathological examination.

Other embodiments with less complicated mechanical linkages may also be used. FIG. 7 depicts a flexible forceps 70, in which a sleeve 71 controls the opening and closing of forceps jaws 72. In this embodiment, jaws 72 may be stamped from a single piece of metal and formed into the desired shape. The jaws are secured to a short cannula 73 and to control rod 75 with a joint 74, which may be formed from a solder joint, a braze joint, a small rivet, or other desired form to secure the jaws to the cannula. Joint 74 could also take the form of a fastener threaded into the cannula to secure the jaws to the cannula. Cannula 73 is secured to control rod or cable 75 by swaging, soldering, brazing or other desired method, including a threaded joint.

Control rod 75 and sleeve 71 are back-loaded into a medical instrument, such as an endoscope or ureteroscope. In this embodiment, jaws 72 when closed are about 4 Fr in diameter, while sleeve 71 containing control rod 75 is about 2.6 Fr in diameter. The larger jaws enable the surgeon to take a larger biopsy sample from a patient in a single sampling procedure, thus taking less time and causing less trauma to the patient. There is a tapered or camming surface 76 to control the opening and closing of the jaws as sleeve 71 is retracted or control rod 75 is advanced.

Another embodiment uses a control rod that separates into two control wires to actuate the grasper or forceps at the distal end. FIG. 8 depicts back-loading grasper having a hollow coil 81, a control rod 83 and an end-effector 82 with a grasper 84. The end-effector is firmly affixed to coil 81 by any suitable method, such as by soldering or brazing. Other methods of attachment may also be used. Grasper 84 at the distal end includes jaws 85 and control links 86 that are connected to wires 83a connected to the control rod. The jaws, even when closed, are too large to be drawn into coil 81, and have a larger diameter or periphery than coil 81. The coil may also have a smooth coating 88, such as a PTFE coating. The grasper is preferably actuated by means of an endoscope or other therapeutic or diagnostic device. The device extends or retracts control rod 83, causing the links 86 to pivot about pivot pin 87, and causing jaws 85 to open or close. The sample or object is thus grasped and removed from the person.

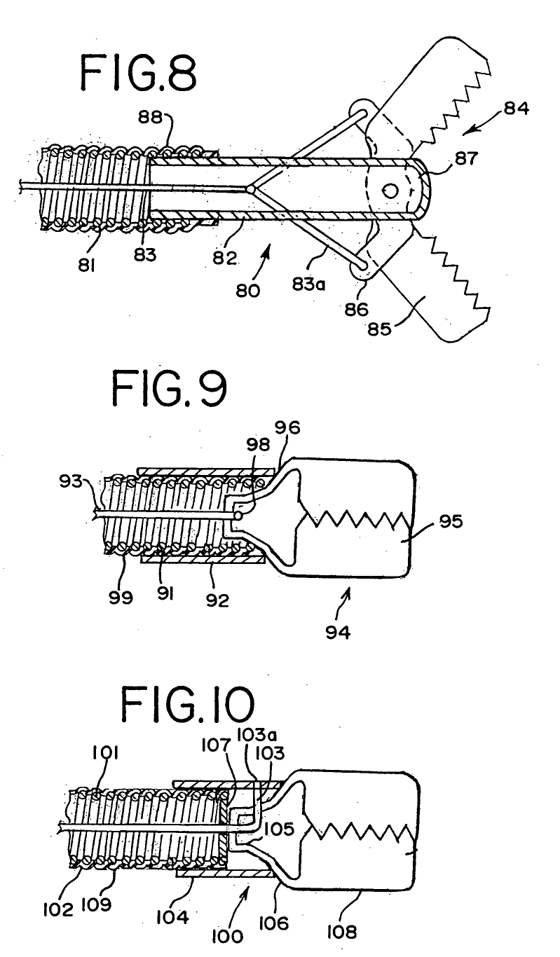

Other embodiments may use a cylinder at the distal end to aid in a sampling procedure using a back-loading instrument. FIGS. 9 and 10 depict such embodiments in a closed position. FIG. 9 depicts a back loading extractor 90 that includes a spring-coil 91. Coil 91 is reinforced on a portion of its distal end with a thin cylinder 92. The cylinder is preferably metal, such as stainless steel, that does not interfere with patient care or with extraction of a sample or of an object from the body. The cylinder may be soldered or brazed to the coil, or otherwise attached firmly so that it will not come off the coil.

The extractor is controlled by a control rod 93 inside the coil, the control rod firmly attached to extractor 94 with jaws 95. The attachment 98 may be made by a braze joint, solder joint, a fastener such as a rivet, or any other suitable joint. Extractor 94 may be made from stainless steel and is preferably made from Nitinol® or other shape memory metal so that the jaws automatically open when control rod 93 urges jaws 95 forward. The extractor has a tapered or camming surface 96, allowing the jaws to spring open as control rod 93 urges jaws 95 forward. Coil 99 and proximal ends (not shown) of the outer coil spring and the control rod may be additionally coated with plastic or other atraumatic coating so that they do not damage an endoscope or other instrument into which the back loading instruments are placed.

FIG. 10 depicts another embodiment of a back-loading endoscopic instrument 100 in the closed position. Instrument 100 includes a coil 101 firmly attached to a grasper 105 with connection 107. Connection 107 may be a braze joint, a solder joint, a fastener, or other convenient joint, so that grasper 105 does not detach from coil 101. Because grasper 105 is brazed or otherwise joined to coil 101, jaws 108 can only open or close, and cannot translate relative to coil 101. Control rod 103 passes through joint 107 and through a slot (not shown) in the proximal portion of grasper 105, allowing the control rod and cylinder to translate relative to coil 101 and grasper 105.

Control rod 103 controls the opening and closing of jaws 108 via a tapered or camming surface 106 of the grasper, using a cylinder 104 attached to control rod 103. The attachment 103a may be any suitable attachment, such as a braze joint, a solder joint, a fastener, or a weld, so that the cylinder does not detach from the control rod. When control rod 103 retracts cylinder 104, jaws 108 open as the camming surfaces 106 allow. When control rod 103 advances, jaws 108 close, following the same camming surfaces.

The outer diameter of cylinder 104 is preferably no more than 2.8 Fr, more preferably about 2.6 Fr, and that of coil spring 101 about 2.4 Fr or less. Grasper 105, camming surfaces 106, and jaws 108 are preferably made from spring steel, stainless steel or a shape memory alloy, such as Nitinol®. The grasper may be stamped from a single continuum of metal and formed into the desired shape. Alternatively, the grasper may be made from several smaller pieces and joined together, by brazing or soldering or by any other suitable method. In this embodiment, the outer diameter of jaws 108 in the closed position is greater than the outer diameter of cylinder 104 and of coil 101. In one embodiment, the outer diameter of the jaws is about 4 Fr. Other embodiments may be smaller or larger, but in any case the outer periphery or circumference of the jaws is greater than the inner diameter of cylinder 104, and the jaws are not able to be retracted into the cylinder.

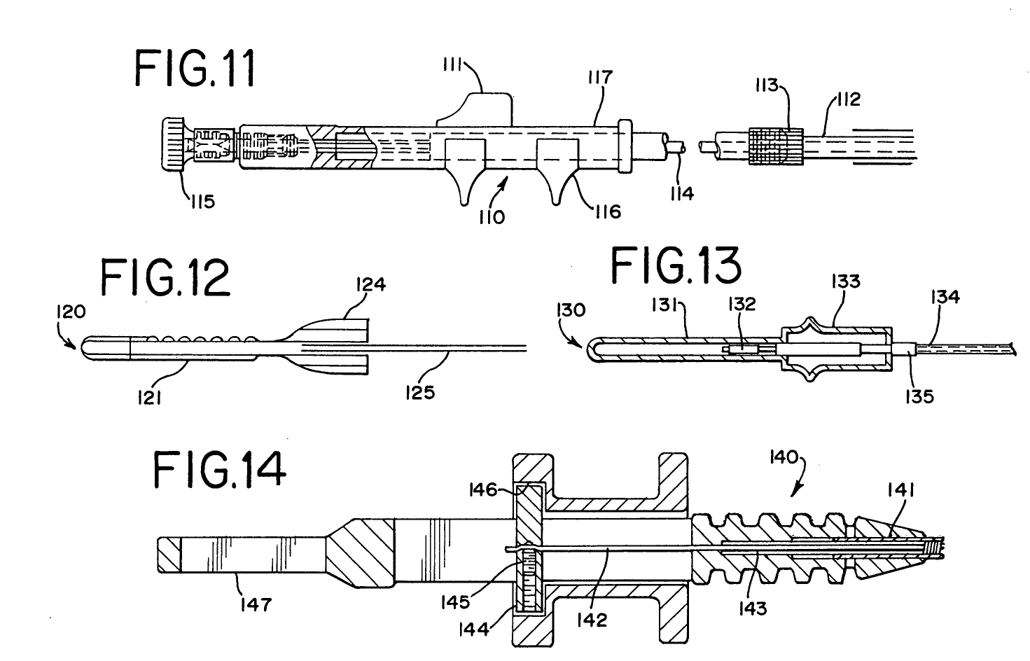

A variety of handles or control devices may be used with embodiments of the endoscopic instruments of the present invention. Handles that may be suitable include those depicted in FIGS. 11-14. FIG. 11 depicts one suitable handle or control device 110. A sheath 112 and control rod 114 from a back-loading endoscopic instrument are attached to the control device 110. In this instance, sheath 112 is fixed to an attachment 113 at a distal end of the control device, while control rod 114 is attached to a movable button 111 for movement by a user. The control rod may be tightened or loosened by knob 115 controlling an internal collet (not shown) or other attachment device. The user controls the device by gripping the handle with the body 117 and fixed finger handles 116 and manipulating control button 111. In other embodiments, the control rod may be fixed and the sheath attached to the collet and movable button.

FIG. 12 depicts a pin vise 120, typically used to control only one of a control rod and sheath. Pin vise 120 may comprise only a handle 121 at its proximal end and a collet 124 at its distal end, used to attach a control element 125 from a back-loading endoscopic instrument. The control element may be a fixed or a movable control rod, or may be a fixed or a movable sheath. Two pin vises may be used, one for a control rod and one for a sheath, the control rod at least partially contained within the sheath.

FIG. 13 depicts another handle or control device 130 for controlling both a fixed and a movable control element. Handle 130 includes a body 131 at a proximal end, a movable control button 133 and a distal end 135. In this embodiment, a sheath 134 from a back-loading endoscopic instrument is fixedly attached to the distal end 135, and a control rod 132 is attached to the movable control button 133. A user controls the endoscopic instrument by manipulating control button 133 back and forth to extend or retract control rod 132. In other embodiments, the sheath may be attached to the movable button and the control rod fixed to the distal end of the handle.

A thumb-ring type handle 140 is depicted in FIG. 14. The handle attached a sheath 141 fixedly and a control rod 142 movably. Control rod 142 is attached to a cross piece 144 by means of a set screw 145. The cross-piece is attached to a movable finger-spool 146. A user holds the handle securely with thumb-ring 147 and controls the device by manipulating movable finger-spool 146 back and forth, extending or retracting control rod 142 and thus operating the back-loading endoscope instrument. In grasper and forceps types of instruments, the control rod may comprise more than a single control rod, e.g., two rods operating two jaws. In other embodiments, the sheath may be movable and the control rod fixed to the distal end of the handle.

It should be understood that the use of larger instruments for back loading is not limited to endoscopes or ureteroscopes. Thus, back-loading instruments may be used on a variety of medical instruments, including hysteroscopes, colonoscopes, gastroscopes, laparoscopes, and so forth. The back-loading instruments may be used in these and in many other applications in which the surgeon wishes to keep trauma and invasiveness to a minimum. Of course, these instruments may also be used in other applications, such as a bronchoscope, in which their small size is still an advantage.

The details of the construction or composition of the various elements of the back loading medical instruments not otherwise disclosed are not believed to be critical to the achievement of the advantages of the present invention, so long as the elements possess the strength, sharpness, and flexibility needed for them to perform as disclosed. The selection of such details of construction is believed to be well within the ability of one of even rudimentary skills in this area, in view of the present disclosure, and are within the spirit of the invention and the scope of the claims. It will be understood that no limitation of the scope of the invention is intended by the above description and drawings, which is defined by the claims below. It is therefore intended that the foregoing detailed description be regarded as illustrative rather than limiting, and that it be understood that it is the following claims, including all equivalents, that are intended to define the spirit and scope of this invention.

1. A back-loading medical instrument for a medical device, the back-loading medical instrument comprising:
a flexible hollow tube;
a control rod within the hollow tube; and
a distal functional portion attached to at least one of the flexible hollow tube and the control rod, the functional portion having a cross-sectional area larger than the tube and larger than a working channel of the medical device.

2. The instrument of claim 1, wherein the functional portion is selected from the group consisting of an end-effector, a biopsy forceps, a hot biopsy forceps, a biopsy mechanism, a cutting mechanism, and a grasping forceps.

3. The instrument of claim 1, wherein the control portion comprises a control cable and a mechanical linkage to the functional portion.

4. The instrument of claim 1, wherein the medical device is selected from the group consisting of an endoscope, a hysteroscope, a ureteroscope, a bronchoscope, a colonoscope, and a gastroscope.

5. The instrument of claim 1, wherein the tube is selected from the group consisting of a coil spring and a polymeric tube.

6. The instrument of claim 1, further comprising an atraumatic proximal end.

7. The instrument of claim 1, further comprising a removable control handle connected to the proximal portion.

8. The instrument of claim 1, wherein the function portion is attached to the control rod and further comprising a cylinder attached to the hollow tube.

9. A back loading instrument for an endoscope, comprising:
a distal portion including a forceps;
a control portion operatively connected to the forceps; and
a proximal portion for insertion into a working channel of an endoscope,
wherein a cross-sectional area of the forceps is greater than a cross-sectional area of a working channel of the endoscope.

10. The instrument of claim 9, wherein the forceps comprises at least one of a cutting portion and a spike.

11. The instrument of claim 9, wherein the distal portion is connected to the control portion by at least one of a crimp joint, a braze joint, a solder joint, a weld joint, and a threaded joint.

12. The instrument of claim 9, wherein the control portion further comprises a smooth exterior coating.

13. A back loading endoscopic biopsy instrument, comprising:
a flexible hollow member having a proximal end and a distal end;
a flexible inner member having a proximal end and a distal end and extending through said hollow outer member; and
a jaw assembly having a pair of jaws extending distally, the jaw assembly coupled to at least one of the hollow outer member and the flexible control member, wherein a circumference of the outer jaws in a closed state is greater than a circumference of a working channel of the endoscope.

14. The instrument of claim 13, wherein the jaw assembly is coupled to the flexible inner member, and the jaw assembly is opened by retracting the flexible hollow member.

15. The instrument of claim 13, wherein the jaw assembly is connected to the flexible hollow member and the flexible inner member, and the jaw assembly is opened or closed by translating the flexible inner member.

16. The instrument of claim 13, further comprising a smooth coating or a thin polymeric sheath on the flexible hollow member.

17. The instrument of claim 13, wherein each of the jaws comprises a cutting edge.

18. A method of making a back loading endoscopic instrument, the method comprising:
forming an openable functional portion, a flexible inner member, and a hollow tubular member;
assembling the hollow tubular member outside the flexible inner member; and
attaching the functional portion to at least one of the flexible inner member and the hollow tubular member, wherein an outer periphery of the openable functional portion is greater than an outer periphery of a working channel of an endoscope when the functional portion is in a closed state.

19. The method of claim 18, further comprising coating at least one of the flexible inner member and the hollow outer member with a smooth plastic coating.

20. The method of claim 18, wherein the functional portion is formed by at least one of machining, stamping, blanking, casting, and electrical discharge machining.

21. A method of using a back-loading medical instrument in a medical device, the method comprising:
providing a back loading medical instrument, wherein the back-loading medical instrument comprises a flexible hollow tube, a control rod within the hollow tube, and a distal functional portion attached to at least one of the flexible hollow tube and the control rod, the functional portion having a cross-sectional area larger than the tube and larger than a working channel of the medical device;
placing the back loading medical instrument into the distal end of the working channel; and
connecting the back loading medical instrument to a control device.

22. The method of claim 21, wherein the back-loading medical instrument is selected from the group consisting of an end-effector, a biopsy forceps, a hot biopsy forceps, a biopsy mechanism, a cutting mechanism, and a grasping forceps.
